(19) United States
(12) Patent Application Publication
(10) Pub. No.: US 2004/0092735 A1
(43) Pub. Date: May 13, 2004

(54) Process for the preparation of cefuroxime sodium

(75) Inventors: Pandurang Balwant Deshpande, Chennai (IN); Pramod Narayan Deshpande, Chennai (IN); Bhausaheb Pandharinath Khadangale, Chennai (IN); Gautam Kumar Das, Chennai (IN); John Muthiah Raja Jeyakumar, Chenagalpattu (IN)

Correspondence Address: OLIFF & BERRIDGE, PLC, P.O. BOX 19928, ALEXANDRIA, VA, 22320, US

(73) Assignee: Orchid Chemicals & Pharmaceuticals Limited, Tamilnadu (IN)

(21) Appl. No.: 10/310,068

(22) Filed: Dec. 5, 2002

(30) Foreign Application Priority Data
Nov. 8, 2002 (IN) 819/MAS/2002

(51) Int. Cl. C07D 501/14

(52) U.S. Cl. 540/222

(57) ABSTRACT

The present invention relates to an improved process for the preparation of the sterile cefuroxime sodium of formula (I).

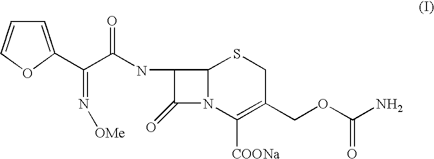

### FIELD OF THE INVENTION

The present invention relates to an improved process for the preparation of active cephalosporin antibiotic derivative. The present invention more particularly relates to an improved process for the preparation of cefuroxime sodium of the formula (I).

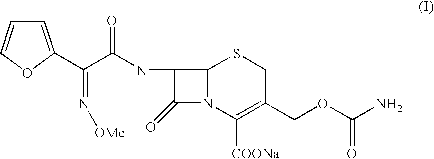

### DESCRIPTION OF THE PRIOR ART

Cefuroxime is a valuable broad spectrum antibiotic and having activity against wide range of gram-positive and gram-negative microorganisms. Cefuroxime sodium is physiologically acceptable non-toxic salt of cefuroxime and may be administered to human or used as a veterinary medicine.

U.S. Pat. No. 4,775,750 discloses the process for the preparation of the compound of formula (I), which involves carbamoylation of (6R,7R)-7-[Z-2-(fur-2-yl)-2-methoxyiminoacetamido]-3-hydroxymethylceph-3-em-4-carboxylic acid in presence of triethylamine and a solvent, and in situ preparation of cefuroxime sodium using sodium-2-ethylhexanote. The product obtained from this patent suffers in non-acceptable color and low purity. Reprocessing is needed to get sterile cefuroxime sodium.

U.S. Pat. No. 4,277,601 describes the process for the preparation of cefuroxime sodium as its THF solvate in situ manner. The process described in this patent involves the usage of multiple organic solvent system and thereby making the process complicated. Moreover, recrystallisation is needed to get the sterile cefuroxime sodium.

The synthesis of cefuroxime disclosed in U.S. Pat. No. 3,966,717 and U.S. Pat. No. 3,974,513 comprises 8 synthetic steps starting from 7-ACA. Such high number of steps, which causes low overall yield, is due to the introduction of two productive groups.

A process for the preparation of cefuroxime starting from 7-ACA has been described in Chemistry and Industry 1984, 217, which do not involve any protective groups. The preparation involves the condensation of 3-hydroxymethyl-7-amino cephalosporanic acid with (fur-2-yl)-2methoxyimino acetic acid using an organic base to produce (6R, 7R)-7-[(Z)-2-(fur-2-yl)-2methoxyiminoacetamido]-3-hydroxymethylceph-3-em-4-carboxylic acid. Carbamoylation of resulting acid with isocyanate of formula RNCO wherein R is a labile substituent to get (6R,7R)-3-carbamoyloxymethyl-7-[Z-2-(fur-2-yl)-2-methoxyiminoacetamido]-ceph-3-em-4-carboxylic acid (cefuroxime).

WO 00/71547 describes a process for the preparation of cefuroxime, which involves enzymatic hydrolysis of 7-glutaryl ACA, which is not industrially viable.

With our continued search and intense investigation, we finally achieved a process for the preparation cefuroxime sodium, which overcomes all difficulties and makes the process industrially viable and yield the title compound in required quantity and quality. In addition, the present invention provides a new process for the conversion of cefuroxime to cefuroxime sodium using a mixture of water soluble sodium salts

### OBJECTIVES OF THE INVENTION

The main objective of the present invention is to provide a process for the preparation of cefuroxime sodium of the formula (I), which has better quality such as color and solubility.

Another objective of the present invention is to provide direct manufacturing process for the preparation of sterile crystalline cefuroxime sodium of the formula (I) from cefuroxime acid.

Still another objective of the present invention is to provide a process for the preparation of cefuroxime sodium of the formula (I) in good yield, high purity and with desirable particle size.

Yet, another objective of the present invention is to provide a simple method for the preparation of cefuroxime of the formula (II) in pure form.

### SUMMARY OF THE INVENTION

Accordingly, the present invention provides an improved process for the preparation of cefuroxime sodium of the formula (I),

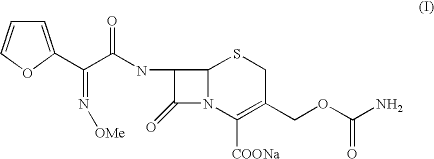

which comprises the steps of:

(i) dissolving the cefuroxime of the formula (II)

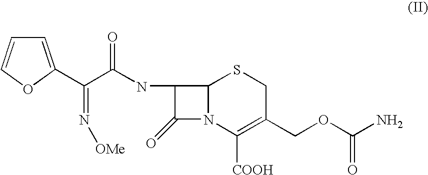

in a water miscible solvent/water at a temperature in the range of 10° C. to 50° C.,

(ii) charcoalising the solution of step (i) followed by micron filtration,

(iii) treating the filtered charcoalized solution of step (ii) with a mixture of water soluble sodium salts of two weak acids in suitable alcoholic solvent at a temperature in the range of 10° C. to 50° C. and

(iv) isolating and drying the precipitated cefuroxime sodium of the formula (I) in pure form.

Another embodiment of the present invention is to provide a process for the preparation of cefuroxime of the formula (II), which comprises the steps of:

(a) condensing 3-hydroxymethyl-7-amino cephalosporanic acid of formula (IV) with activated (fur-2-yl)-2-methoxyimino acetic acid of formula (V) in the presence of an inorganic base in a solvent at a temperature in the range of −40° C. to 10° C. and isolating the product by adjusting the pH to 1.5 to 2.5 using an acid to produce (6R,7R)-7-[(Z)-2-(fur-2-yl)-2-methoxyiminoacetamido]-3-hydroxymethylceph-3-em-4-carboxylic acid of formula (VI) and

(b) carbamoylating the compound of formula (VI) with isocyanate of formula (VII) wherein R is a labile group in the presence of a solvent at a temperature in the range of −60° C. to 0° C. to get cefuroxime of the formula (II).

The process is shown in scheme-I as given below:

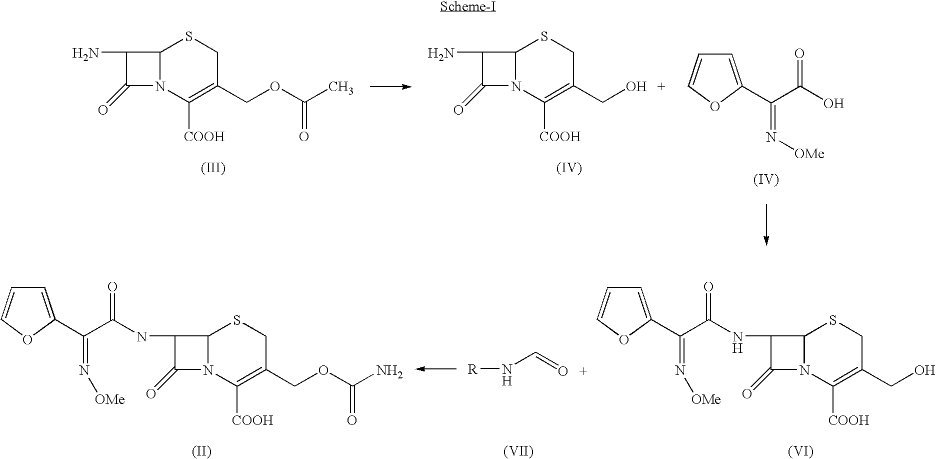

### DETAILED DESCRIPTION OF THE INVENTION

In an embodiment of the present invention, cefuroxime of formula (II) was dissolved in water miscible solvent/water such as acetone, THF, acetonitrile at a temperature in the range of 10° C. to 50° C.

In still another embodiment of the present invention, the mixture of water soluble sodium salt of weak acid is selected from sodium lactate/sodium acetate, sodium 2-ethyl hexonate/sodium acetate and the like. The reaction may be carried out in an alcoholic solvent selected from methanol, ethanol, isopropyl alcohol or mixtures thereof, at a temperature in the range of 10° C. to 50° C. The advantage of using the mixture of water soluble sodium salt of two weak acids is that the yield is higher and the color of the product obtained is better.

In still another embodiment of the present invention, the compound of formula (I) was isolated from reaction mass by adding suitable water miscible solvent such as acetone, methanol, ethanol, isopropyl alcohol or mixtures thereof.

In yet another embodiment of the present invention, 7-amino cephalosporanic acid of formula (III) was dissolved in solvents such as methanol, acetone, dichloromethane, THF, water or mixtures thereof, to that sodium hydroxide solution was added at a temperature in the range of −90° C. to 0° C., to produce compound of formula (IV).

In still another embodiment of the present invention, the activation of fur-2-yl methoxyimino acetic acid of formula (V) is carried out using PCl₅, DMF/POCl₃, oxalyl chloride, SOCl₂/DMF, diphenylchlorophosphoridate, dialkyl chlorophosphoridate, in the presence of a solvent selected from halogenated alkanes, ethyl acetate, tetrahydrofuran, aromatic hydrocarbons, acetone, acetonitrile, dialkylethers or mixtures thereof at a temperature in the range of −40° C. to 10° C.

In still another embodiment of present invention the compound of formula (IV) was reacted with the active derivative of fur-2-yl methoxyimino acetic acid of formula (V) in step (a), in the presence of inorganic base such as sodium bicarbonate, sodium hydroxide, potassium carbonate or sodium carbonate in the presence of solvents such as methanol, acetone, dichloromethane, THF, water or mixtures thereof to produce the compound of formula (VI).

In yet another embodiment of the present invention, the acid used for adjusting pH in step (a) is selected from hydrochloric acid, sulphuric acid or ortho phosphoric acid.

In yet another embodiment of the present invention, the labile group represented by R is selected from chlorosulphonyl, mono, di or trichloroacetyl, bromosulphonyl, trichloro ethoxycarbonyl, trimethylsilyl or chlorobenzenesulphonyl group.

In still another embodiment of the present invention the compound of formula (VI) was reacted with the compound of formula (VII) to produce the compound of formula (II) in step (b), in the presence of a solvent selected from THF, methanol, dichloromethane, acetone, water or mixtures thereof.

In one more embodiment of the present invention the cefuroxime sodium of the formula (I) obtained is sterile crystalline syn isomer.

The present invention is exemplified by the following examples, which is provided for illustration only and should not be construed to limit the scope of the invention.

### EXAMPLE (1)

Step (i): Preparation of (6R,7R)-7-[(Z)-2-(fur-2-yl)-2-methoxyiminoacetamido]-3-hydroxymethylceph-3-em-4-carboxylic Acid.

(i) To a mixture of dimethyl acetamide (145 ml), dichloromethane (22 ml) and dimethylformamide (28 ml) in a dry flask, (fur-2-yl)-2-methoxyimino acetic acid ammonium salt (73 gm) was added and cooled the reaction mass to −40° C. To the reaction mass POCl₃ (60 gm) was added at −40° C. and stirred the reaction mass at −20±2° C. for 45 minutes. The mass was cooled to −30° C. and kept at that temperature for condensation.

(ii) To a mixture of water (400 ml) and methanol (400 ml) in another flask, 7-amino cephalosporinic acid (100 gm) was added and cooled the slurry to −50° C. To the reaction mass sodium hydroxide solution (29 gm NaOH in 200 ml water) was added at 50° C. and stirred for 60 minutes at 40±2° C. After completion of reaction the pH of reaction was adjusted to 7.0 to 8.0 using dilute HCl. Temperature of this reaction mass was raised to 0° C. by the addition of saturated sodium bicarbonate solution (800 ml) followed by (fur-2-yl)-2-methoxyimino acetic acid mass from step (i) at 0-2° C. After completion of reaction, pH was adjusted to 2.0 using dilute HCl (80 ml). The product formed was filtered, washed with water followed by dichloromethane and dried the product under vacuum at 45° C. to get the title compound of formula (VI) (116-118 gm-) in pure form.

Step (ii): Preparation of (6R,7R)-3-carbamoyloxymethyl-7-[(Z)-2-(fur-2-yl)-2-methoxyiminoacetamido]-ceph-3-em-4-carboxylic Acid (Cefuroxime Acid):

(6R,7R)-7-[Z-2-(Fur-2-yl)-2-methoxyiminoacetamido]-3-hydroxymethyl ceph-3-em-4-carboxylic acid (100 gm) obtained from step (i) was dissolved in THF (480 ml) at 0° C. and cooled to −50° C. To the cooled solution chlorosulphonyl isocyanate (59 gm in 100 ml THF) solution was added at −50° C. and stirred at the same temperature for 60 minutes. The reaction mass was poured into precooled water at 10° C. Stirred the mass at 10° C. till completion of reaction. The reaction mass was washed with ethyl acetate and then aqueous layer was subjected to charcoal treatment. The pH of aqueous solution was adjusted to 2.0 using dilute HCl. The product formed was filtered and washed with water followed by isopropyl alcohol to produce the title compound of formula (II) (105 gm).

Step (iii) Preparation of (6R,7R)-3-carbamoyloxymethyl-7-[(Z)-2-(fur-2-yl)-2-methoxyiminoacetamido]-ceph-3-em-4-carboxylic Acid Sodium (Cefuroxime Sodium):

(6R,7R)-3-carbamoyloxymethyl-7-[Z-2-(fur-2-yl)-2-methoxyimino acetamido]-ceph-3-em-4-carboxylic acid (100 gm) was dissolved in a mixture of acetone (650 ml) and water (800 ml) at 25° C. Activated carbon was added and stirred for 15 minutes at 25° C. The carbon was filtered and washed the bed with acetone/water. The solution was then passed through series of micron filters in a sterile area. The solution was warmed to 35° C. A mixture of sodium lactate solution (23 gm) (60% solution in water) and sodium acetate (16.5 gm) in ethanol (25 ml) was added to the reaction mixture slowly at 35° C. and stirred for 30 minutes. To the thick slurry acetone (900 ml) was charged at 35° C. The product obtained was filtered and washed with ethanol followed by acetone. The product was dried under vacuum to get sterile cefuroxime sodium (98 gm) in pure form.

### EXAMPLE (2)

Preparation of (6R,7R)-3-carbamoyloxymethyl-7-[Z2-(fur-2-yl)-2-methoxy iminoacetamido]-ceph-3-em-4-carboxylic Acid Sodium (Cefuroxime Sodium):

(6R,7R)-3-carbamoyloxymethyl-7-[Z-2-(fur-2-yl)-2-methoxyimino acetamido]-ceph-3-em-4-carboxylic acid (100 gm) was dissolved in a mixture of acetone (650 ml)/water (800 ml) at 25° C. Activated carbon was added and stirred for 15 minutes at 25° C. The carbon was filtered and washed the bed with acetone/water. The solution was then passed through series of micron filters in a sterile area. The solution was warmed to 35° C. A mixture of sodium lactate solution (23 gm) (60% solution in water) and sodium acetate (16.5 gm) in methanol (450 ml) was added to the reaction mixture slowly at 35° C. and stirred for 30 minutes. The product obtained was filtered and washed with methanol (200 ml) followed by acetone. The product was dried under vacuum to get sterile cefuroxime sodium (98 gm) in pure form.

1) A process for the preparation of the cefuroxime sodium of the formula (I), said 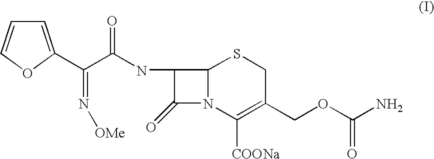
process comprising the steps of:
(i) dissolving the cefuroxime of the formula (II) 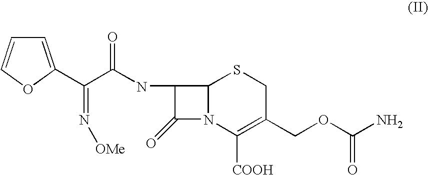
in a water miscible solvent/water at a temperature in the range of 10° C. to 50° C.,
(ii) charcoalising the solution of step (i) followed by micron filtration,
(iii) treating the filtered charcoalized solution of step (ii) with a mixture of water soluble sodium salts of two weak acids in suitable alcoholic solvent at a temperature in the range of 10° C. to 50° C., and
(iv) isolating and drying the precipitated compound of formula (i) in pure form.

2) The process as claimed in claim 1, wherein the water miscible solvent used in step (i) is selected from acetone, tetrahydrofuran (THF) and acetonitrile or mixtures thereof.

3) The process as claimed in claim 1, wherein a mixture of water-soluble sodium salts used in step (iii) is selected from sodium lactate/sodium acetate or sodium 2-ethyl hexanoate/sodium acetate.

4) The process as claimed in claim 1, wherein the alcoholic solvent used in step (iii) is selected from methanol, ethanol and isopropyl alcohol or mixtures thereof.

5) The process as claimed in claim 1, wherein the solvent used for isolation in step (iv) is acetone, methanol, ethanol and isopropyl alcohol or mixtures thereof.

6) The process as claimed in claim 1, wherein the compound of formula (I) is in crystalline form.

7) The process as claimed in claim 1, wherein the compound of formula (I) is a sterile product.

8) The process as claimed in claim 1, wherein the compound of formula (I) is a syn isomer.

9) A process for the preparation of cefuroxime of the formula (II) 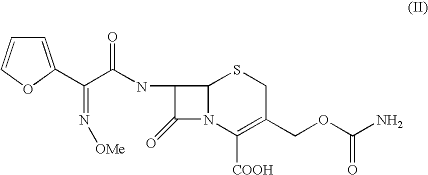
the said process comprising the steps of:
(a) condensing 3-hydroxymethyl-7-amino cephalosporanic acid of the formula (IV) 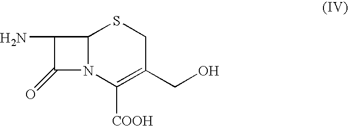
with activated (fur-2-yl)-2-methoxyimino acetic acid of the formula (V) 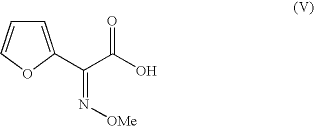
in the presence of an inorganic base in a solvent at a temperature in the range of −40° C. to 10° C. and isolating the product by adjusting the pH to 1.5 to 2.5 using an acid to produce (6R,7R)-7-[(Z)-2-(fur-2-yl)-2-methoxyiminoacetamido]-3-hydroxymethyl ceph-3-em-4carboxylic acid of the formula (VI), 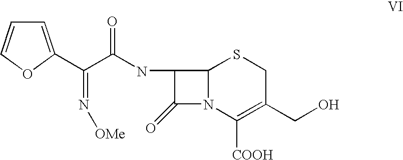
(b) carbamoylating the compound of formula (VI) with isocyanate of formula (VII)
RNCO (VII)
wherein R is a labile group at a temperature in the range of −60° C. to 0° C. to get cefuroxime acid of the formula (II).

10) The process as claimed in claim 9, wherein the base used in step (a) is selected from sodium hydroxide, sodium bicarbonate, potassium carbonate and sodium carbonate.

11) The process as claimed in claim 9, wherein the solvent used in step (a) is selected from methanol, acetone, dichloromethane, THF and water or mixtures thereof.

12) The process as claimed in claim 9, wherein the acid used in step (a) is selected from hydrochloric acid, sulphuric acid and ortho phosphoric acid.

13) The process as claimed in claim 9, wherein the solvent used in step (b) is selected from methanol, acetone, dichloromethane, THF and water or mixtures thereof.

14) The process as claimed in claim 9, wherein the labile group represented by R is selected from chlorosulphonyl, mono, di or trichloroacetyl, bromosulphonyl, trichloroethoxycarbonyl, trimethylsilyl or chlorobenzenesulphonyl group.
